(19) United States
(12) Patent Application Publication
(10) Pub. No.: US 2006/0233721 A1
(43) Pub. Date: Oct. 19, 2006

(54) Foam containing unique oil globules

(75) Inventors: Dov Tamarkin, Maccabim (IL); Doron Friedman, Karmei Yosef (IL); Alex Besonov, Rehovot (IL); Meir Eini, Ness Ziona (IL)

Correspondence Address: WILMER CUTLER PICKERING HALE AND DORR LLP, 60 STATE STREET, BOSTON, MA, 02109, US

(73) Assignee: Foamix Ltd., Rehovot (IL)

(21) Appl. No.: 11/389,742

(22) Filed: Mar. 27, 2006

Related U.S. Application Data
(63) Continuation-in-part of application No. 10/911,367, filed on Aug. 4, 2004.
(63) Continuation-in-part of application No. 10/532,618, filed on Dec. 22, 2005.
(60) Provisional application No. 60/492,385, filed on Aug. 4, 2003.
(60) Provisional application No. 60/429,546, filed on Nov. 29, 2002.
(60) Provisional application No. 60/717,058, filed on Sep. 14, 2005.

(30) Foreign Application Priority Data
Oct. 25, 2002 (IL) 152486

(51) Int. Cl. A61K 8/36

(52) U.S. Cl. 424/47; 977/926

(57) ABSTRACT

The present invention provides a foamable composition for administration to the skin, body surface, body cavity or mucosal surface, e.g., the mucosa of the nose, mouth, eye, ear, respiratory system, vagina or rectum. The foamable oil in water emulsion composition includes: an oil globule system, selected from the group consisting of oil bodies; and sub-micron oil globules, about 0.1% to about 5% by weight of an agent, selected from the group consisting of a surface-active agent, having an HLB value between 9 and 16; and a polymeric agent, and a liquefied or compressed gas propellant at a concentration of about 3% to about 25% by weight of the total composition, water and optional ingredients are added to complete the total mass to 100%. Upon release from an aerosol container, the foamable composition forms and expanded foam suitable for topical administration.

### CROSS REFERENCE TO RELATED APPLICATIONS

This application is a continuation-in-part application of co-pending U.S. patent application Ser. No.10/532,618, filed on Apr. 25, 2005, which is an application filed under 35 U.S.C. §371 of International Patent Application No. IB03/005527 designating the United States and filed on Oct. 24, 2003, which claims the benefit of priority under 35 U.S.C. §119(e) to U.S. Patent Application Ser. No. 60/429,546, filed on Nov. 29, 2002, both entitled “Cosmetic and Pharmaceutical Foam,” and which also claims the benefit of priority under 35 USC§119(a) to Israeli Patent Appl. No. 152486, filed Oct. 25, 2002, all of which are hereby incorporated in their entirety by reference.

This application is a continuation-in-part application of co-pending U.S. patent application Ser. No.10/911,367, filed on Aug. 4, 2004, which claims the benefit of priority under 35 U.S.C. §119(e) to U.S. Patent Application Ser. No. 60/492,385, filed on Aug. 4, 2003, both entitled “Foam Carrier Containing Amphiphilic Copolymer Gelling Agent” and both hereby incorporated in their entirety by reference.

This application claims priority under 35 U.S.C. §119(e) to co-pending U.S. Provisional Application No. 60/717,058, filed Sep. 14, 2005, entitled “Foam Containing Unique Oil Globules,” which is incorporated in its entirety by reference.

### BACKGROUND OF THE INVENTION

Foams and, in particular, foam emulsions are complex dispersion systems which do not form under all circumstances. Slight shifts in foam emulsion composition, such as by the addition of active ingredients, may destabilize the foam.

Microemulsions and nanoemulsion are monophasic, transparent (or slightly translucent) dispersions of oil and water. Unlike conventional emulsions, microemulsions and nanoemulsion are thermodynamically stable, making them a favorable vehicle for pharmaceutical compositions, which have to maintain stability for long periods of time.

Storage triacylglycerols (TAG) in plant seeds are present in small discrete intracellular organelles ranging from 1 to 2 μm, which are called oil-bodies. An oil body has a matrix of TAG, which is surrounded by phospholipids (PL) and alkaline proteins, termed oleosins. Oleosins are highly lipophilic proteins, are expressed at high levels in many seeds and are specifically targeted to oil-bodies. Oil-bodies are abundant in plant seeds and are among the simplest organelles present in eukaryotes. They are remarkably stable both inside the cells and in isolated preparations.

Oil bodies are also termed in the literature as “oleosomes”, “lipid bodies” and “spherosomes”.

U.S. Pat. Nos. 5,683,710 and 5,613,583 disclose emulsions comprising lipid vesicles from oleaginous plants.

U.S. Pat. Nos. 6,777,591, 6,753,167, 6,599,513, 6,596,287, 6,582,710, 6,372,234, 6,210,742, 6,183,762, 6,146,645, 5,948,682, 5,856,452, 5,792,922 and 5,650,554 describe various products comprising oil bodies, including products for topical applications.

U.S. Pat. No. 5,679,324 pertains to an aerosol foamable fragrance composition, translucent in its pre-dispensed state, which forms a fast-breaking foam. The composition contains a surfactant selected from the group consisting of ethoxylated lanolin oil derivatives, propoxylated lanolin oil derivatives, and mixtures thereof, a propellant, a fragrance, a thickener, and a cosmetic vehicle (preferably water).

U.S. Pat. No. 6,730,288 teaches a pharmaceutical foam composition including (a) an active ingredient; (b) an occlusive agent; (c) an aqueous solvent; and (d) an organic cosolvent; wherein the active ingredient is insoluble in water and insoluble in both water and the occlusive agent; and wherein there is enough occlusive agent to form an occlusive layer on the skin.

### SUMMARY OF THE INVENTION

In one aspect of the present invention there is provided a foamable oil in water emulsion, composition containing small oil globules including an oil globule system, selected from the group consisting of oil bodies and sub-micron oil globules, about 0.1% to about 5% by weight of at least one stabilizing agent selected from the group consisting of a non-ionic surface-active agent having an HLB value between 9 and 16, an ionic surfactant, and a polymeric agent water, as well as a liquefied or compressed gas propellant at a concentration of about 3% to about 25% by weight of the total composition.

According to further embodiments of the foamable composition of present invention, the oil globule system consists of oil bodies and the stabilizing agent consists of a polymeric agent.

According to still further embodiments of the foamable composition of present invention, the oil globule system consists of oil bodies and the stabilizing agent consists of an ionic surfactant.

According to yet further embodiments of the present invention the surface active agent is a phospholipid.

According to still further embodiments of the present invention, the oil bodies are discrete oleaginous particles ranging from about 1 to about 3 μm in dimension. Oil bodies contain triacylclycerols (TAG), surrounded by phospholipids (PL) and oleosins.

According to further embodiments of the present invention, the phospholipids are selected from the group consisting of phosphatidylethanolamine, phosphatidylcholine, lecithin, phosphatidylserine, phosphatidylglycerol and phosphatidylinositol.

According to still further embodiments of the present invention, the oleosins are highly lipophilic small proteins of about 25 to 26 kD.

In one or more embodiments, the oil bodies are derived from the seeds of a plant, selected from the group consisting of almond (Prunus dulcis), anise (Pimpinella anisum), avocado (Persea spp.), beach nut (Fagus sylvatica), borage (also known as evening primrose) (Boragio officinalis), Brazil nut (Bertholetia excelsa), candle nut (Aleuritis tiglium), carapa (Carapa guineensis), cashew nut (Ancardium occidentale), castor (Ricinus communis), coconut (Cocus nucifera), coriander (Coriandrum sativum), cottonseed (Gossypium spp.), crambe (Crambe abyssinica), Crepis alpina, croton (Croton tiglium), Cuphea spp., dill (Anethum gravealis), Euphorbia lagascae, Dimorphoteca pluvialis, false flax (Camolina sativa), fennel (Foeniculum vulgaris), groundnut (Arachis hypogaea), hazelnut (coryllus avellana), hemp (Cannabis sativa), honesty plant (lunnaria annua), jojoba (Simmiondsia chinensis), kapok fruit (Ceiba pentandra), kukui nut (Aleuritis moluccana), Lesquerella spp., linseed/flax (Linum usitatissimum), macademia nut (Macademia spp.), maize (Zea mays), meadow foam (Limnanthes alba), mustard (Brassica spp. and Sinapis alba), oil palm (Elaeis guineeis), oiticia (Licania rigida), paw paw (Assimina triloba), pecan (Juglandaceae ssp.), perilla (Perilla futescens), physic nut (Gairopha curcas), pilinut (Canariuim ovatum), pine nut (pine spp.), pistachio (Pistachia vera), pongam (Bongamin glabra), poppy seed (Papaver soniferum), rapeseed (Brassica spp.), safflower (Carthamus tinctorius), sesame seed (Sesamum indicum), soybean (Glycine max), squash (Cucurbita maxima), sal tree (Shorea rubusha), Stokes aster (Stokesia laevis), sunflower (Helianthus annuus), tukuma (Astocarya spp.), tung nut (Aleuritis cordata), and vernolnia (Verzonia galamensis).

According to a further embodiment of the foamable composition, the foamable composition further includes about 0.1% to about 5% by weight of a foam adjuvant selected from the group consisting of a fatty alcohol having 15 or more carbons in their carbon chain, a fatty acid having 16 or more carbons in their carbon chain, fatty alcohols derived from beeswax and including a mixture of alcohols, a majority of which has at least 20 carbon atoms in their carbon chain, a fatty alcohol having at least one double bond, a fatty acid having at least one double bond, a branched fatty alcohol, a branched fatty acid, and a fatty acid substituted with a hydroxyl group and mixtures thereof.

According to further embodiments of the present invention, the foamable composition is substantially alcohol-free.

According to still further embodiments of the present invention, the concentration range of oil globules is selected from the group of (i) about 0.05% and about 2% and about 5%, (ii) about 2% (iii) about 5% and about 12%, and (iv) about 12% and about 24%.

According to further embodiments of the present invention, the polymeric agent is selected from the group consisting of a water-soluble cellulose ether and naturally-occurring polymeric material.

According to still further embodiments of the present invention, the water-soluble cellulose ether is selected from the group consisting of methylcellulose, hydroxypropyl cellulose, hydroxypropyl methylcellulose (Methocel), hydroxyethyl cellulose, methylhydroxyethylcellulose, methylhydroxypropylcellulose, hydroxyethylcarboxymethylcellulose, carboxymethylcellulose, carboxymethylhydroxyethylcellulose, xanthan gum, guar gum, carrageenin gum, locust bean gum and tragacanth gum.

According to yet further embodiments of the present invention, the foamable composition further includes at least one therapeutic agent.

According to further embodiments of the present invention, the therapeutic agent is selected from the group consisting of an anti-infective, an antibiotic, an antibacterial agent, an antifungal agent, an antiviral agent, an antiparasitic agent, an steroidal anti-inflammatory agent, an immunosuppressive agent, an immunomodulator, an immunoregulating agent, a hormonal agent, vitamin A, a vitamin A derivative, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a disinfectant, an anesthetic, an antiallergic agent, an alpha hydroxyl acid, lactic acid, glycolic acid, a beta-hydroxy acid, a protein, a peptide, a neuropeptide, a allergen, an immunogenic substance, a haptene, an oxidizing agen, an antioxidant, a dicarboxylic acid, azelaic acid, sebacic acid, adipic acid, fumaric acid, a retinoid, an antiproliferative agent, an anticancer agent, a photodynamic therapy agent, an anti-wrinkle agent, a radical scavenger, a metal oxide (e.g., titanium dioxide, zinc oxide, zirconium oxide, iron oxide, silicone oxide, an anti wrinkle agent, a skin whitening agent, a skin protective agent, a masking agent, an anti-wart agent, a refatting agent, a lubricating agent and mixtures thereof.

According to still further embodiments of the present invention, the therapeutic agent is selected from the components of the oil bodies or sub-micron oil globules.

According to further embodiments of the present invention, the therapeutic agent is suitable to treat a disorder selected from the group consisting of dermatological disorder, a cosmetic disorder, a gynecological disorder, a disorder of a body cavity, wound and burn.

According to a further embodiment of the present invention, there is provided a method of treating, alleviating or preventing a disorder of the skin, body cavity or mucosal surface, wherein the disorder involves insufficient hydration of skin or a mucosal surface as one of its etiological factors. The method includes administering topically to a subject having the disorder, a foamed composition containing an oil globule system selected from the group consisting of oil bodies and sub-micron oil globules, about 0.1% to about 5% by weight of at least one stabilizing agent selected from the group consisting of a non-ionic surface-active agent having an HLB value between 9 and 16, an ionic surfactant, and a polymeric agent, water, and a liquefied or compressed gas propellant at a concentration of about 3% to about 25% by weight of the total composition.

According to a further embodiment of the method, the composition further includes an active agent effective to treat a disorder, and wherein the disorder is selected from the group consisting of a vaginal disorder, a vulvar disorder, an anal disorder, a disorder of a body cavity, an ear disorder, a disorder of the nose, a disorder of the respiratory system, a bacterial infection, fungal infection, viral infection, dermatosis, dermatitis, parasitic infections, disorders of hair follicles and sebaceous glands, scaling papular diseases, benign tumors, malignant tumors, reactions to sunlight, bullous diseases, pigmentation disorders, disorders of cornification, pressure sores, disorders of sweating, inflammatory reactions, xerosis, ichthyosis, allergy, burn, wound, cut, chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, osteoarthritis, joint pain, hormonal disorder, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum.

According to a further embodiment of the present invention, there is provided a method to promote the penetration of an active agent into the surface layers of the skin and mucosal membranes. The method includes applying a foamable composition to the surface layers of a skin or mucosal membrane the foamable composition, comprising an oil globule system selected from the group consisting of oil bodies and sub-micron oil globules, about 0.1% to about 5% by weight of at least one stabilizing agent selected from the group consisting of a non-ionic surface-active agent having an HLB value between 9 and 16, an ionic surfactant, and a polymeric agent, water, and a liquefied or compressed gas propellant at a concentration of about 3% to about 25% by weight of the total composition.

### DETAILED DESCRIPTION OF THE INVENTION

The present invention provides a foamable oil in water emulsion, composition including small oil globules. As used herein, the terms droplets, globules and particles, when referencing an emulsion, are used interchangeably. All % values are provided on a weight (w/w) basis.

According to one or more embodiments of the present invention, the foamable oil in water emulsion composition is intended for administration to the skin, a body surface, a body cavity or mucosal surface, e.g., the mucosa of the nose, mouth, eye, ear, respiratory system, vagina or rectum (severally and interchangeably termed herein “target site”). The foamable oil in water emulsion composition includes:
(a) an oil globule system selected from the group consisting of oil bodies and sub-micron oil globules;
(b) about 0.1% to about 5% by weight of at least one stabilizing agent selected from the group consisting of a non-ionic surface-active agent selected from the group consisting of a non-ionic surface-active agent, having an HLB value between 9 and 16, an ionic surfactant; and a polymeric agent; and
(c) a liquefied or compressed gas propellant at a concentration of about 3% to about 25% by weight of the total composition.

Water and optional ingredients are added to complete the total mass to 100%. Upon release from an aerosol container, the foamable composition forms an expanded foam suitable for topical administration.

In one or more embodiments, the oil globules are oil bodies. Oil bodies, also termed “oleosomes”, “lipid bodies” and “spherosomes”, are small discrete oleaginous particles, ranging in size from about 1 to about 3 μm along one dimension. Oil bodies consist of triacylglycerols (TAG) surrounded by phospholipids (PL) and alkaline proteins, termed oleosins.

Triacylglycerides (also termed triglycerides) are chemically defined as glycerol esters of fatty acids. The seed oil present in the oil body fraction of plant species is a mixture of various triacylglycerides, of which the exact composition depends on the plant species from which the oil is derived.

Phospolipids possess a structure that is very similar to that of the triacylglycerides except that a terminal carbon of the glycerol backbone is esterified to phosphoric acid. Substitution of the hydrogen atom of phosphatidic acid results in additional phospholipids classes, including but not limited to the following:

| Substitution | Phospholipid |
|---|---|
| Ethanolamine | Phosphatidylethanolamine |
| Choline | Phosphatidylcholine, also called lecithins |
| Serine | Phosphatidylserine |
| Glycerol | Phosphatidylglycerol |
| Myo-inositol | Phosphatidylinositol |

Oleosins are highly lipophilic small proteins of about 15 to 26 kD. They are expressed at high levels in many seeds and are specifically targeted to oil-bodies. Oleosins completely cover the surface of the subcellular oil bodies.

Oil-bodies are abundant in plant seeds and are among the simplest organelles present in eukaryotes. They are remarkably stable both inside the cells and in isolated preparations.

Oil bodies are prepared from plant seeds. Exemplary plant seeds include (alphabetically) almond (Prunus dulcis); anise (Pimpinella anisum); avocado (Persea spp.); beach nut (Fagus sylvatica); borage (also known as evening primrose) (Boragio officinalis); Brazil nut (Bertholletia excelsa); candle nut (Aleuritis tiglium); carapa (Carapa guineensis); cashew nut (Ancardium occidentale); castor (Ricinus communis); coconut (Cocus nucifera); coriander (Coriandrum sativum); cottonseed (Gossypium spp.); crambe (Crambe abyssinica); Crepis alpina; croton (Croton tiglium); Cuphea spp.; dill (Anethum gravealis); Euphorbia lagascae; Dimorphoteca pluvialis; false flax (Camolina sativa); fennel (Foeniculum vulgaris); groundnut (Arachis hypogaea); hazelnut (coryllus avellana); hemp (Cannabis sativa); honesty plant (Lunnaria annua); jojoba (Simmiondsia chinensis); kapok fruit (Ceiba pentandra); kukui nut (Aleuritis moluccana); Lesquerella spp., linseed/flax (Linum usitatissimum); macademia nut (Macademia spp.); maize (Zea mays); meadow foam (Limnanthes alba); mustard (Brassica spp. and Sinapis alba); oil palm (Elaeis guineeis); oiticia (Licania rigida); paw paw (Assimina triloba); pecan (Juglandaceae spp.); perilla (Perilla frutescens); physic nut (Gairopha curcas); pilinut (Canariuim ovatum); pine nut (pine spp.); pistachio (Pistachia vera); pongam (Bongamin glabra); poppy seed (Papaver soniferum); rapeseed (Brassica spp.); safflower (Carthamus tinctorius); sesame seed (Sesamum indicum); soybean (Glycine max); squash (Cucurbita maxima); sal tree (Shorea rubusha); Stokes aster (Stokesia laevis); sunflower (Helianthus annuus); tukuma (Astocarya spp.); tung nut (Aleuritis cordata); and vernolnia (Verzonia galamensis). Isolation of oil bodies from plant sources is well known. See, for example, U.S. Pat. No. 5,650,554.

Stable artificial oil bodies can be reconstituted with triacylglycerol, phospholipid, and oleosin via sonication, as described, for example in J. T. C. Tzen, Y. Z. Cao, P. Laurent, C. Ratnayake, and A. H. C. Huang. 1993. Lipids, proteins, and structure of seed oil bodies from diverse species. Plant Physiol. 101:267-276.

The skin-beneficial effects of oil bodies include, but are not limited to (1) antioxidant effects (resulting from the presence of tocopherol and other antioxidants naturally present in the oil bodies); (2) occlusivity, as determined by improved skin barrier function and reduced trans-epidermal water loss; and (3) emolliency. Furthermore, the oil bodies building blocks—the triacylglycerides and the phospholipids—contain unsaturated or polyunsaturated fatty acids. Exemplary unsaturated fatty acids are omega-3 and omega-6 fatty acids. Other examples of such polyunsaturated fatty acids are linoleic and linolenic acid, gamma-linoleic acid (GLA), eicosapentaenoic acid (EPA) and docosahexaenoic acid (DHA). Such unsaturated fatty acids are known for their skin-conditioning and anti-inflammatory effects, which contribute to the therapeutic benefit of the present foamable composition.

Because oil bodies contain phospholipids and oleosins, which concurrently carry hydrophobic and hydrophilic moieties, they act as emulsifiers and, as a result, upon dilution with water with mild mixing, they spontaneously form an emulsion.

In one or more embodiments, the oil globules are sub-micron oil globules, i.e., oil globules, which have a number-average size of less than 1,000 nm. An emulsion, comprising sub-micron globules or nano-size globules is called sub-micron emulsion (“SME”) or microemulsion or nanoemulsion, respectively. In one or more embodiments, the oil globules have a number-average size of less than 500 nm; or less than 200 nm; or less than 100 nm. In certain embodiments, the oil globules have number-average size in the following ranges: (i) 40 nm to 1,000 nm. (ii) 40 nm to 500 nm; (iii) 40 nm to 200 nm; or (iv) 40 nm to 100 nm.

SMEs are dispersions of oil and water. With reference to conventional emulsions, SMEs are more stable, making them a favorable vehicle for pharmaceutical compositions, which have to maintain stability for long periods of time. SMEs may be used in vehicles for transporting nutraceuticals, medicaments, peptides or proteins. The decrease in size of the globules makes it possible to promote the penetration of the active agents into the surface layers of the skin and mucosal membranes.

In SMEs, the active compounds can be solubilized. The general concept of solubilization of active components and its utilization may be found in the following review articles: 1. Solans, C., Pons, R., Kunieda, H “Overview of basic aspects of microemulsions” Industrial Applications of Microemulsions, Solans, C., Kunieda, H., Eds.: Dekker, New York (1997); 66: 1-17, 2. Dungan, S. R., “Microemulsions in foods: properties and application” ibid 148-170; 3. Holmberg, K. “Quarter century progress and new horizons in microemulsions” in Micelles, Microemulsions and Monolayers, Shah, O. Ed.; Dekker: New York (1998) 161-192; 4. Garti, N. “Microemulsions, emulsions, double emulsions and emulsions in food” in Formulation Science (proceeding from formulation forum '97 association of formulation chemists) (1998) 1, 147-219; 5. Ezrahi, S., Aserin, A. Garti, N. in Micoremulsions-fundamental wad applied aspects Kumar, P. and Mittal, K. L. Eds. Marcel Dekker, Inc. New York (1999) “Aggregation behavior in one-phase (Winsor IV) systems” 185-246; 6. Garti, N. Clement, V., Leser, M., Aserin, A. Fanun, M. “Sucrose esters microemulsions J. Molec. Liquids (1999) 80, 253-296.

In certain embodiments, the production of SMEs and nanoemulsion involves very-high sheer homogenizers. An exemplary homogenizer, suitable for producing nano-emulsions is the commercially-available “Microfluidizer®”. Microfluidizer® fluid processors are built for deagglomeration and dispersion of uniform submicron particles and creation of stable emulsions and dispersions. Microfluidizer processors overcome limitations of conventional processing technologies by utilizing high-pressure streams that collide at ultra-high velocities in precisely defined microchannels. Combined forces of shear and impact act upon products to attain uniform particle and droplet size reduction (often submicron), deagglomeration and high yield cell disruption.

Notwithstanding the above, any other very-high sheer homogenizer, capable of producing submicron particles is suitable for use in the production of a microemulsions or a nanoemulsion according to the present invention.

In additional embodiments, the SMEs form spontaneously with gentle mixing such as hand shaking.

The sub-micron particles contain at least one organic carrier, preferably a hydrophobic organic carrier. In addition, the composition may contain one or more of a hydrophobic organic carrier, a polar solvent, an emollient and mixtures thereof, at a concentration of about 2% to about 5%, or about 5% to about 10%, or about 10% to about 20%, or about 20% to about 50% by weight.

A “hydrophobic organic carrier” as used herein refers to a material having solubility in distilled water at ambient temperature of less than about 1 gm per 100 mL, more preferable less than about 0.5 gm per 100 mL, and most preferably less than about 0.1 gm per 100 mL. It is liquid at ambient temperature. The identification of a hydrophobic organic carrier or “hydrophobic solvent”, as used herein, is not intended to characterize the solubilization capabilities of the solvent for any specific active agent or any other component of the foamable composition. Rather, such information is provided to aid in the identification of materials suitable for use as a hydrophobic carrier in the foamable compositions described herein.

In one or more embodiments, the hydrophobic organic carrier is an oil, such as mineral oil. Mineral oil (Chemical Abstracts Service Registry number 8012-95-1) is a mixture of aliphatic, naphthalenic, and aromatic liquid hydrocarbons that derive from petroleum. It is typically liquid; its viscosity is in the range of between about 35 CST and about 100 CST (at 40° C.), and its pour point (the lowest temperature at which an oil can be handled without excessive amounts of wax crystals forming so preventing flow) is below 0° C. In one or more embodiments, the term hydrophobic organic carrier does not include thick or semi-solid materials, such as white petrolatum, also termed “Vaseline”, which, in certain compositions is disadvantageous due to its waxy nature and semi-solid texture.

According to one or more embodiments, hydrophobic solvents are liquid oils originating from vegetable, marine or animal sources. Suitable liquid oil includes saturated, unsaturated or polyunsaturated oils. By way of example, the unsaturated oil may be olive oil, corn oil, soybean oil, canola oil, cottonseed oil, coconut oil, sesame oil, sunflower oil, borage seed oil, syzigium aromaticum oil, hempseed oil, herring oil, cod-liver oil, salmon oil, flaxseed oil, wheat germ oil, evening primrose oils or mixtures thereof, in any proportion.

Suitable hydrophobic solvents also include polyunsaturated oils containing poly-unsaturated fatty acids. In one or more embodiments, said unsaturated fatty acids are selected from the group of omega-3 and omega-6 fatty acids. Examples of such polyunsaturated fatty acids are linoleic and linolenic acid, gamma-linoleic acid (GLA), eicosapentaenoic acid (EPA) and docosahexaenoic acid (DHA). Such unsaturated fatty acids are known for their skin-conditioning effect, which contribute to the therapeutic benefit of the present foamable composition. Thus, the hydrophobic solvent can include at least 6% of an oil selected from omega-3 oil, omega-6 oil, and mixtures thereof. In the context of the present invention, oils that possess therapeutically beneficial properties are termed “therapeutically active oil.”

Another class of hydrophobic solvents is the essential oils, which are also considered therapeutically active oil, which contain active biologically occurring molecules and, upon topical application, exert a therapeutic effect, which is conceivably synergistic to the beneficial effect of the NSAID in the composition.

Another class of therapeutically active oils includes liquid hydrophobic plant-derived oils, which are known to possess therapeutic benefits when applied topically.

Silicone oils also may be used and are desirable due to their known skin protective and occlusive properties. Suitable silicone oils include non-volatile silicones, such as polyalkyl siloxanes, polyaryl siloxanes, polyalkylaryl siloxanes and polyether siloxane copolymers, polydimethylsiloxanes (dimethicones) and poly(dimethylsiloxane)-(diphenyl-siloxane) copolymers. These are chosen from cyclic or linear polydimethylsiloxanes containing from about 3 to about 9, preferably from about 4 to about 5, silicon atoms. Volatile silicones such as cyclomethicones can also be used. Silicone oils are also considered therapeutically active oil, due to their barrier retaining and protective properties.

The organic carrier may be a mixture of two or more of the above hydrophobic solvents in any proportion.

A further class of organic carriers includes “emollients” that have a softening or soothing effect, especially when applied to body areas, such as the skin and mucosal surfaces. Emollients are not necessarily hydrophobic. Examples of suitable emollients include hexyleneglycol, propylene glycol, isostearic acid derivatives, isopropyl palmitate, isopropyl isostearate, diisopropyl adipate, diisopropyl dimerate, maleated soybean oil, octyl palmitate, cetyl lactate, cetyl ricinoleate, tocopheryl acetate, acetylated oil bodies alcohol, cetyl acetate, phenyl trimethicone, glyceryl oleate, tocopheryl linoleate, wheat germ glycerides, arachidyl propionate, myristyl lactate, decyl oleate, propylene glycol ricinoleate, isopropyl lanolate, pentaerythrityl tetrastearate, neopentylglycol dicaprylate/dicaprate, isononyl isononanoate, isotridecyl isononanoate, myristyl myristate, triisocetyl citrate, octyl dodecanol, sucrose esters of fatty acids, octyl hydroxystearate and mixtures thereof.

According to one or more embodiments of the present invention, the organic carrier includes a mixture of a hydrophobic solvent and an emollient. According to one or more embodiments, the foamable composition is a mixture of mineral oil and an emollient in a ratio between 2:8 and 8:2 on a weight basis.

A “polar solvent” is an organic solvent, typically soluble in both water and oil. Examples of polar solvents include polyols, such as glycerol (glycerin), propylene glycol, hexylene glycol, diethylene glycol, propylene glycol n-alkanols, terpenes, di-terpenes, tri-terpenes, terpen-ols, limonene, terpene-ol, 1-menthol, dioxolane, ethylene glycol, other glycols, sulfoxides, such as dimethylsulfoxide (DMSO), dimethylformanide, methyl dodecyl sulfoxide, dimethylacetamide, monooleate of ethoxylated glycerides (with 8 to 10 ethylene oxide units), azone (1-dodecylazacycloheptan-2-one), 2-(n-nonyl)-1,3-dioxolane, esters, such as isopropyl myristate/palmitate, ethyl acetate, butyl acetate, methyl proprionate, capric/caprylic triglycerides, octylmyristate, dodecyl-myristate; myristyl alcohol, lauryl alcohol, lauric acid, lauryl lactate ketones; amides, such as acetamide oleates such as triolein; various alkanoic acids such as caprylic acid; lactam compounds, such as azone; alkanols, such as dialkylamino acetates, and admixtures thereof.

According to one or more embodiments, the polar solvent is a polyethylene glycol (PEG) or PEG derivative that is liquid at ambient temperature, including PEG200 (MW (molecular weight) about 190-210 kD), PEG300 (MW about 285-315 kD), PEG400 (MW about 380-420 kD), PEG600 (MW about 570-630 kD) and higher MW PEGs such as PEG 4000, PEG 6000 and PEG 10000 and mixtures thereof.

According to one or more embodiments, the foamable composition is substantially alcohol-free, i.e., free of short chain alcohols. Short chain alcohols, having up to 5 carbon atoms in their carbon chain skeleton and one hydroxyl group, such as ethanol, propanol, isopropanol, butanol, iso-butanol, t-butanol and pentanol, are considered less desirable solvents or polar solvents due to their skin-irritating effect. Thus, the composition is substantially alcohol-free and includes less than about 5% final concentration of lower alcohols, preferably less than about 2%, more preferably less than about 1%.

The composition includes a stabilizing agent, which may be a polymeric agent. The polymeric agent serves to stabilize the foam composition and to control drug residence in the target organ. Exemplary polymeric agents are classified below in a non-limiting manner. In certain cases, a given polymer can belong to more than one of the classes provided below.

The polymeric agent may be a gelling agent. A gelling agent controls the residence of a therapeutic composition in the target site of treatment by increasing the viscosity of the composition, thereby limiting the rate of its clearance from the site. Many gelling agents are known in the art to possess mucoadhesive properties.

The gelling agent can be a natural gelling agent, a synthetic gelling agent and an inorganic gelling agent. Exemplary gelling agents that can be used in accordance with one or more embodiments of the present invention include, for example, naturally-occurring polymeric materials, such as locust bean gum, sodium alginate, sodium caseinate, egg albumin, gelatin agar, carrageenin gum, sodium alginate, xanthan gum, quince seed extract, tragacanth gum, guar gum, starch, chemically modified starches and the like, semi-synthetic polymeric materials such as cellulose ethers (e.g. hydroxyethyl cellulose, methyl cellulose, carboxymethyl cellulose, hydroxy propylmethyl cellulose), guar gum, hydroxypropyl guar gum, soluble starch, cationic celluloses, cationic guars, and the like, and synthetic polymeric materials, such as carboxyvinyl polymers, polyvinylpyrrolidone, polyvinyl alcohol, polyacrylic acid polymers, polymethacrylic acid polymers, polyvinyl acetate polymers, polyvinyl chloride polymers, polyvinylidene chloride polymers and the like. Mixtures of the above compounds are contemplated.

Further exemplary gelling agents include the acrylic acid/ethyl acrylate copolymers and the carboxyvinyl polymers sold, for example, by the B.F. Goodrich Company under the trademark of Carbopol® resins. These resins consist essentially of a colloidal water-soluble polyalkenyl polyether crosslinked polymer of acrylic acid crosslinked with from 0.75% to 2% of a crosslinking agent such as polyallyl sucrose or polyallyl pentaerythritol. Examples include Carbopol® 934, Carbopol® 940, Carbopol® 950, Carbopol® 980, Carbopol® 951 and Carbopol® 981. Carbopol® 934 is a water-soluble polymer of acrylic acid crosslinked with about 1% of a polyallyl ether of sucrose having an average of about 5.8 allyl groups for each sucrose molecule.

The gelling agent may be a water-soluble cellulose ether. Preferably, the water-soluble cellulose ether is selected from the group consisting of methylcellulose, hydroxypropyl cellulose, hydroxypropyl methylcellulose (Methocel), hydroxyethyl cellulose, methylhydroxyethylcellulose, methylhydroxypropylcellulose, hyd roxyethylcarboxymethylcel lu lose, carboxymethylcel lu lose and carboxymethylhydroxyethylcellulose. More preferably, the water-soluble cellulose ether is selected from the group consisting of methylcellulose, hydroxypropyl cellulose and hydroxypropyl methylcellulose (Methocel). In one or more embodiments, the composition includes a combination of a water-soluble cellulose ether; and a naturally-occurring polymeric materials, selected from the group including xanthan gum, guar gum, carrageenan gum, locust bean gum and tragacanth gum.

Yet, in other embodiments, the gelling agent includes inorganic gelling agents, such as silicone dioxide (fumed silica).

The polymeric agent may be a mucoadhesive agent. Mucoadhesion/bioadhesion is defined as the attachment of synthetic or biological macromolecules to a biological tissue. Mucoadhesive agents are a class of polymeric biomaterials that exhibit the basic characteristic of a hydrogel, i.e. swell by absorbing water and interacting by means of adhesion with the mucous that covers epithelia. Compositions of the present invention may contain a mucoadhesive macromolecule or polymer in an amount sufficient to confer bioadhesive properties. The bioadhesive macromolecule enhances the delivery of biologically active agents on or through the target surface. The mucoadhesive macromolecule may be selected from acidic synthetic polymers, preferably having at least one acidic group per four repeating or monomeric subunit moieties, such as poly(acrylic)—and/or poly(methacrylic) acid (e.g., Carbopol®, Carbomer®)), poly(methylvinyl ether/maleic anhydride) copolymer, and their mixtures and copolymers; acidic synthetically modified natural polymers, such as carboxymethylcellulose (CMC); neutral synthetically modified natural polymers, such as (hydroxypropyl)methylcellulose; basic amine-bearing polymers such as chitosan; acidic polymers obtainable from natural sources, such as alginic acid, hyaluronic acid, pectin, gum tragacanth, and karaya gum; and neutral synthetic polymers, such as polyvinyl alcohol or their mixtures. An additional group of mucoadhesive polymers includes natural and chemically modified cyclodextrin, especially hydroxypropyl-β-cyclodextrin. Such polymers may be present as free acids, bases, or salts, usually in a final concentration of about 0.01% to about 0.5% by weight.

A suitable bioadhesive macromolecule is the family of acrylic acid polymers and copolymers, (e.g., Carbopol®). These polymers contain the general structure—[CH₂—CH(COOH)—]ₙ. Hyaluronic acid and other biologically-derived polymers may be used.

Exemplary bioadhesive or mucoadhesive macromolecules have a molecular weight of at least 50 kDa, or at least 300 kDa, or at least 1,000 kDa. Favored polymeric ionizable macromolecules have not less than 2 mole percent acidic groups (e.g., COOH, SO3H) or basic groups (NH2, NRH, NR2), relative to the number of monomeric units. The acidic or basic groups can constitute at least 5 mole percent, or at least 10 mole percent, or at least 25, at least 50 more percent, or even up to 100 mole percent relative to the number of monomeric units of the macromolecule.

Yet, another group of mucoadhesive agent includes inorganic gelling agents such as silicon dioxide (fumed silica), including but not limited to, AEROSIL 200 (DEGUSSA).

Many mucoadhesive agents are known in the art to also possess gelling properties.

The polymeric agent may be a film forming component. The film forming component may include at least one water-insoluble alkyl cellulose or hydroxyalkyl cellulose. Exemplary alkyl cellulose or hydroxyalkyl cellulose polymers include ethyl cellulose, propyl cellulose, butyl cellulose, cellulose acetate, hydroxypropyl cellulose, hydroxybutyl cellulose, and ethylhydroxyethyl cellulose, alone or in combination. In addition, a plasticizer or a cross linking agent may be used to modify the polymer's characteristics. For example, esters such as dibutyl or diethyl phthalate, amides such as diethyldiphenyl urea, vegetable oils, fatty acids and alcohols such as oleic and myristyl acid may be used in combination with the cellulose derivative.

The polymeric agent may be a phase change polymer, which alters the composition behavior from fluid-like prior to administration to solid-like upon contact with the target mucosal surface. Such phase change results from external stimuli, such as changes in temperature or pH and exposure to specific ions (e.g., Ca²⁺). Non-limiting examples of phase change polymers include poly(N-isopropylamide), Poloxamer 407® and Smart-Gel® (Poloxamer+PAA). The polymeric agent is present in an amount in the range of about 0.01% to about 5.0% by weight of the foam composition. In one or more embodiments, it is typically less than about 1 wt % of the foamable composition.

The stabilizing agent may also be a surface active agent. Surface-active agents (also termed “surfactants”) include any agent linking oil and water in the composition, in the form of emulsion. A surfactant's hydrophilic/lipophilic balance (HLB) describes the emulsifier's affinity toward water or oil. The HLB scale ranges from 1 (totally lipophilic) to 20 (totally hydrophilic), with 10 representing an equal balance of both characteristics. Lipophilic emulsifiers form water-in-oil (w/o) emulsions; hydrophilic surfactants form oil-in-water (o/w) emulsions. The HLB of a blend of two emulsifiers equals the weight fraction of emulsifier A times its HLB value plus the weight fraction of emulsifier B times its HLB value (weighted average).

According to one or more embodiments of the present invention, the surface-active agent has a hydrophilic lipophilic balance (HLB) between about 9 and about 16, which is the required HLB (the HLB required to stabilize an O/W emulsion of a given oil) of most oils and hydrophobic solvents. Thus, in one or more embodiments, the composition contains a single surface active agent having an HLB value between about 9 and 16, and in one or more embodiments, the composition contains more than one surface active agent and the weighted average of their HLB values is between about 9 and about 16.

The surface-active agent is selected from anionic, cationic, nonionic, zwitterionic, amphoteric and ampholytic surfactants, as well as mixtures of these surfactants. Such surfactants are well known to those skilled in the therapeutic and cosmetic formulation art. Nonlimiting examples of possible surfactants include polysorbates, such as polyoxyethylene (20) sorbitan monostearate (Tween 60) and poly(oxyethylene) (20) sorbitan monooleate (Tween 80); poly(oxyethylene) (POE) fatty acid esters, such as Myrj 45, Myrj 49, Myrj 52 and Myrj 59; poly(oxyethylene) alkylyl ethers, such as poly(oxyethylene) cetyl ether, poly(oxyethylene) palmityl ether, polyethylene oxide hexadecyl ether, polyethylene glycol cetyl ether, brij 38, brij 52, brij 56 and brij W1; sucrose esters, partial esters of sorbitol and its anhydrides, such as sorbitan monolaurate and sorbitan monolaurate; mono or diglycerides, isoceteth-20, sodium methyl cocoyl taurate, sodium methyl oleoyl taurate, sodium lauryl sulfate, triethanolamine lauryl sulfate and betaines.

In one or more embodiments of the present invention, the surface-active agent includes at least one non-ionic surfactant. Ionic surfactants are known to be irritants. Therefore, non-ionic surfactants are preferred in applications including sensitive tissue such as found in most mucosal tissues, especially when they are infected or inflamed. We have surprisingly found that non-ionic surfactants alone provide foams of excellent quality, i.e. a score of “E” according to the grading scale discussed herein below.

In one or more embodiments, the surface active agent includes a mixture of at least one non-ionic surfactant and at least one ionic surfactant in a ratio in the range of about 100:1 to 6:1. In one or more embodiments, the non-ionic to ionic surfactant ratio is greater than about 6:1, or greater than about 8:1; or greater than about 14:1, or greater than about 16:1, or greater than about 20:1.

In one or more embodiments of the present invention, a combination of a non-ionic surfactant and an ionic surfactant (such as sodium lauryl sulfate and cocamidopropylbetaine) is employed, at a ratio of between 1:1 and 20:1, or at a ratio of 4:1 to 10:1. The resultant foam has a low specific gravity, e.g., less than 0.1 g/ml.

It has been surprisingly discovered that the stability of the composition is especially pronounced when a combination of at least one non-ionic surfactant having HLB of less than 9 and at least one non-ionic surfactant having HLB of equal or more than 9 is employed. The ratio between the at least one non-ionic surfactant having HLB of less than 9 and the at least one non-ionic surfactant having HLB of equal or more than 9, is between 1:8 and 8:1, or at a ratio of 4:1 to 1:4. The resultant HLB of such a blend of at least two emulsifiers is between about 9 and about 16.

Thus, in an exemplary embodiment, a combination of at least one non-ionic surfactant having HLB of less than 9 and at least one non-ionic surfactant having HLB of equal or more than 9 is employed, at a ratio of between 1:8 and 8:1, or at a ratio of 4:1 to 1:4, wherein the HLB of the combination of emulsifiers is between about 9 and about 16.

In one or more embodiments of the present invention, the surface-active agent includes mono-, di- and tri-esters of sucrose with fatty acids (sucrose esters), prepared from sucrose and esters of fatty acids or by extraction from sucro-glycerides. Suitable sucrose esters include those having high monoester content, which have higher HLB values.

In the case wherein the oil globules are oil bodies, the surface active agent can be the phospholipids or the oil bodies.

Combination of surface active agents are contemplated. The total surface active agent is in the range of about 0.1 to about 5% of the foamable composition, and is typically less than about 2% or less than about 1%.

Preferably, foam adjuvant is included in the foamable compositions of the present invention to increase the foaming capacity of surfactants and/or to stabilize the foam. In one or more embodiments of the present invention, the foam adjuvant agent includes fatty alcohols having 15 or more carbons in their carbon chain, such as cetyl alcohol and stearyl alcohol (or mixtures thereof). Other examples of fatty alcohols are arachidyl alcohol (C20), behenyl alcohol (C22), 1-triacontanol (C30), as well as alcohols with longer carbon chains (up to C50). Fatty alcohols, derived from beeswax and including a mixture of alcohols, a majority of which has at least 20 carbon atoms in their carbon chain, are especially well suited as foam adjuvant agents. The amount of the fatty alcohol required to support the foam system is inversely related to the length of its carbon chains. Foam adjuvants, as defined herein are also useful in facilitating improved spreadability and absorption of the composition.

In one or more embodiments of the present invention, the foam adjuvant agent includes fatty acids having 16 or more carbons in their carbon chain, such as hexadecanoic acid (C16) stearic acid (C18), arachidic acid (C20), behenic acid (C22), octacosanoic acid (C28), as well as fatty acids with longer carbon chains (up to C50), or mixtures thereof. As for fatty alcohols, the amount of fatty acids required to support the foam system is inversely related to the length of its carbon chain.

Optionally, the carbon atom chain of the fatty alcohol or the fatty acid may have at least one double bond. A further class of foam adjuvant agent includes a branched fatty alcohol or fatty acid. The carbon chain of the fatty acid or fatty alcohol also can be substituted with a hydroxyl group, such as 12-hydroxy stearic acid.

An important property of the fatty alcohols and fatty acids used in context of the composition of the present invention is related to their therapeutic properties per se. Long chain saturated and mono unsaturated fatty alcohols, e.g., stearyl alcohol, erucyl alcohol, arachidyl alcohol and behenyl alcohol (docosanol) have been reported to possess antiviral, antiinfective, antiproliferative and antiinflammatory properties (see, for example, U.S. Pat. No. 4,874,794). Longer chain fatty alcohols, e.g., tetracosanol, hexacosanol, heptacosanol, octacosanol, triacontanol, etc., are also known for their metabolism modifying properties and tissue energizing properties. Long chain fatty acids have also been reported to possess anti-infective characteristics.

Thus, in preferred embodiments of the present invention, a combined and enhanced therapeutic effect is attained by including both a nonsteroidal immunomodulating agent and a therapeutically effective foam adjuvant in the same composition, thus providing a simultaneous anti-inflammatory and antiinfective effect from both components. Furthermore, in a further preferred embodiment, the composition concurrently comprises a nonsteroidal immunomodulating agent, a therapeutically effective foam adjuvant and a therapeutically active oil, as detailed above. Such combination provides an even more enhanced therapeutic benefit. Thus, the foamable carrier, containing the foam adjuvant provides an extra therapeutic benefit in comparison with currently used vehicles, which are inert and non-active.

The foam adjuvant according to preferred embodiments of the present invention includes a mixture of fatty alcohols, fatty acids and hydroxy fatty acids and derivatives thereof in any proportion, providing that the total amount is 0.1% to 5% (w/w) of the carrier mass. More preferably, the total amount is 0.4% -2.5% (w/w) of the carrier mass.

The foam of the present invention may further optionally include a variety of formulation excipients, which are added in order to fine-tune the consistency of the formulation, protect the formulation components from degradation and oxidation and modify their consistency. Such excipients may be selected, for example, from stabilizing agents, antioxidants, humectants, preservatives, colorant and odorant agents and other formulation components, used in the art of formulation.

Aerosol propellants are used to generate and administer the foamable composition as a foam. The total composition including propellant, foamable compositions and optional ingredients is referred to as the foamable carrier. The propellant makes up about 3% to about 25% of the foamable carrier. Examples of suitable propellants include volatile hydrocarbons such as butane, propane, isobutane or mixtures thereof, and fluorocarbon gases.

Cosmetically or Pharmaceutically Active Agents

In one or more embodiments, the foamable composition of the present invention is a carrier of a cosmetically or pharmaceutically active agent(s). The agents may be introduced into an aqueous phase (i.e., water), or a hydrophobic phase (e.g., hydrophobic solvent or oil globules). Exemplary, non binding and cosmetically or pharmaceutically active agents include, but are not limited to an anti-infective, an antibiotic, an antibacterial agent, an antifungal agent, an antiviral agent, an antiparasitic agent, an steroidal antiinflammatory agent, an immunosuppressive agent, an immunomodulator, an immunoregulating agent, a hormonal agent, vitamin A, a vitamin A derivative, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a disinfectant, an anesthetic, an antiallergic agent, an alpha hydroxyl acid, lactic acid, glycolic acid, a beta-hydroxy acid, a protein, a peptide, a neuropeptide, a allergen, an immunogenic substance, a haptene, an oxidizing agent, an antioxidant, a dicarboxylic acid, azelaic acid, sebacic acid, adipic acid, fumaric acid, a retinoid, an antiproliferative agent, an anticancer agent, a photodynamic therapy agent, an anti-wrinkle agent, a radical scavenger, a metal oxide (e.g., titanium dioxide, zinc oxide, zirconium oxide, iron oxide), silicone oxide, an anti wrinkle agent, a skin whitening agent, a skin protective agent, a masking agent, an anti-wart agent, a refatting agent, a lubricating agent and mixtures thereof. Yet, in certain embodiments, one or more components of the oil bodies or sub-micron globules act possess a therapeutic property, such as detailed hereinabove, and thus, in such embodiments, the oil bodies or sub-micron globules can be considered herein as active agents.

Composition and Foam Physical Characteristics

A pharmaceutical or cosmetic composition manufactured using the foam carrier according to one or more embodiments of the present invention is very easy to use. When applied onto the afflicted body surface of mammals, i.e., humans or animals, it is in a foam state, allowing free application without spillage. Upon further application of a mechanical force, e.g., by rubbing the composition onto the body surface, it freely spreads on the surface and is rapidly absorbed.

The foam composition of the present invention creates a stable formulation having an acceptable shelf-life of at least one year, or at least two years at ambient temperature. A feature of a product for cosmetic or medical use is long term stability. Propellants, which are a mixture of low molecular weight hydrocarbons, tend to impair the stability of emulsions. It has been observed, however, that foam compositions according to the present invention are surprisingly stable. Following accelerated stability studies, they demonstrate desirable texture; they form fine bubble structures that do not break immediately upon contact with a surface, spread easily on the treated area and absorb quickly.

The composition should also be free flowing, to allow it to flow through the aperture of the container, e.g., and aerosol container, and create an acceptable foam.

Foam Quality can be Graded as Follows:

Grade E (excellent): very rich and creamy in appearance, does not show any bubble structure or shows a very fine (small) bubble structure; does not rapidly become dull; upon spreading on the skin, the foam retains the creaminess property and does not appear watery;

Grade G (good): rich and creamy in appearance, very small bubble size, “dulls” more rapidly than an excellent foam, retains creaminess upon spreading on the skin, and does not become watery;

Grade FG (fairly good): a moderate amount of creaminess noticeable, bubble structure is noticeable; upon spreading on the skin the product dulls rapidly and becomes somewhat lower in apparent viscosity;

Grade F (fair): very little creaminess noticeable, larger bubble structure than a “fairly good” foam, upon spreading on the skin it becomes thin in appearance and watery;

Grade P (poor): no creaminess noticeable, large bubble structure, and when spread on the skin it becomes very thin and watery in appearance; and

Grade VP (very poor): dry foam, large very dull bubbles, difficult to spread on the skin.

Topically administratable foams are typically of quality grade E or G, when released from the aerosol container. Smaller bubbles are indicative of more stable foam, which does not collapse spontaneously immediately upon discharge from the container. The finer foam structure looks and feels smoother, thus increasing its usability and appeal.

A further aspect of the foam is breakability. The foam of the present invention is thermally stable, yet breaks under sheer force. Sheer-force breakability of the foam is clearly advantageous over thermally-induced breakability. Thermally sensitive foams immediately collapse upon exposure to skin temperature and, therefore, cannot be applied on the hand and afterwards delivered to the afflicted area.

Another property of the foam is density (specific gravity), as measured upon release from the aerosol can. Typically, foams have specific gravity of (1) less than 0.12 g/mL; or (2) the range between 0.02 and 0.12; or (3) the range between 0.04 and 0.10; or (4) the range between 0.06 and 0.10.

Fields of Pharmaceutical Applications

By including oil bodies or sub-micron globules and optionally, additional active agents in the compositions of the present invention, the composition are useful in treating an animal or a human patient having any one of a variety of dermatological disorders that include dry and/or scaly skin as one or their etiological factors (also termed “dermatoses”), such as classified in a non-limiting exemplary manner according to the following groups:

Dermatitis including contact dermatitis, atopic dermatitis, seborrheic dermatitis, nummular dermatitis, chronic dermatitis of the hands and feet, generalized exfoliative dermatitis, stasis dermatitis; lichen simplex chronicus; diaper rash;

Bacterial infections including cellulitis, acute lymphangitis, lymphadenitis, erysipelas, cutaneous abscesses, necrotizing subcutaneous infections, staphylococcal scalded skin syndrome, folliculitis, furuncles, hidradenitis suppurativa, carbuncles, paronychial infections, erythrasma;

Fungal Infections including dermatophyte infections, yeast Infections; parasitic Infections including scabies, pediculosis, creeping eruption;

Viral Infections;

Disorders of hair follicles and sebaceous glands including acne, rosacea, perioral dermatitis, hypertrichosis (hirsutism), alopecia, including male pattern baldness, alopecia areata, alopecia universalis and alopecia totalis; pseudofolliculitis barbae, keratinous cyst;

Scaling papular diseases including psoriasis, pityriasis rosea, lichen planus, pityriasis rubra pilaris;

Benign tumors including moles, dysplastic nevi, skin tags, lipomas, angiomas, pyogenic granuloma, seborrheic keratoses, dermatofibroma, keratoacanthoma, keloid;

Malignant tumors including basal cell carcinoma, squamous cell carcinoma, malignant melanoma, paget's disease of the nipples, kaposi's sarcoma;

Reactions to sunlight including sunburn, chronic effects of sunlight, photosensitivity;

Bullous diseases including pemphigus, bullous pemphigoid, dermatitis herpetiformis, linear immunoglobulin A disease;

Pigmentation disorders including hypopigmentation such as vitiligo, albinism and postinflammatory hypopigmentation and hyperpigmentation such as melasma (chloasma), drug-induced hyperpigmentation, postinflammatory hyperpigmentation;

Disorders of cornification including ichthyosis, keratosis pilaris, calluses and corns, actinic keratosis;

Pressure sores;

Disorders of sweating; and

Inflammatory reactions including drug eruptions, toxic epidermal necrolysis; erythema multiforme, erythema nodosum, granuloma annulare.

According to one or more embodiments of the present invention, the compositions are also useful in the therapy of non-dermatological disorders by providing transdermal delivery of an active nonsteroidal immunomodulating agent that is effective against non-dermatological disorders.

The same advantage is expected when the composition is topically applied to a body cavity or mucosal surface (e.g., the mucosa of the nose, mouth, eye, ear, vagina or rectum) to treat conditions such as chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum.

The following examples exemplify the pharmacological compositions and methods described herein. The examples are for the purposes of illustration only and are not intended to be limiting of the invention.

### EXAMPLE 1

### SME-Based Foamable Composition

1. Emulsion Formula

| | | % w/w |
|---|---|---|
| A | Mineral oil (oil) | 5.60 |
| | Isopropyl myristate (emollient) | 5.60 |
| | Glyceryl monostearate (emollient) | 0.45 |
| | PEG-40 Stearate (surface active agent) | 2.60 |
| | Stearyl alcohol (foam adjuvant) | 0.85 |
| B | Xanthan gum (gelling agent) | 0.26 |
| | Methocel K100M (gelling agent) | 0.26 |
| | Polysorbate 80 (surface-active agent) | 0.90 |
| | Water | 74.88 |
| C | Preservative | 0.60 |
| D | Propellant | 8.00 |
| | | 100.00 |

2. Emulsion Preparation

Oil Phase (A): The ingredients of the Oil Phase were preheated to the same temperature, e.g., 40-75° C., and then were combined with mixing. Oil soluble cosmetic or pharmaceuticals active ingredients and optional oil soluble formulation ingredients are added with agitation to the Oil Phase mixture.

Aqueous Phase (B): Water gelling agent and surface-active agent were dissolved in water, with agitation. The solution was warmed to 50-70° C. Water soluble cosmetic or pharmaceutical active ingredients and optional water soluble ingredients were added with agitation to the Aqueous Phase mixture.

The warm Oil Phase was gradually poured into the warm Aqueous Phase, with agitation, followed by Ultraturax homogenization. The mixture was allowed to cool down to ambient temperature. In case of heat sensitive active ingredients, the active ingredient can be added with agitation to the mixture after cooling to ambient temperature. The mixture, at ambient temperature, was added to an aerosol container, the container was sealed and appropriate amount of propellant (5-25 w % of the composition mass) was added under pressure into the container.

Microscopic observation of the resulting emulsion revealed mean particle size of 2 to 4 microns.

3. Conversion of the Emulsion to Nanoemulsion (Pre-Foam Composition)

The emulsion was passed through a microfluidizer, Microfluidics M-110Y Microfluidizer®) about 10 cycles, using ice to avoid heating the formula.

4. Packaging and Dressurizing of the Nanoemulsion Composition

A nanoemulsion composition (46 gram) was introduced into a 60 ml monoblock aluminum can. The can was closed with an aerosol valve and 4 gram of liquefied propellant (propane butane isobutene mixture) was added through the valve.

5. Characterization of the Nanoemulsion

Particle size distribution was determined using a Malvern nanosizer™ instrument. The pre-foam composition showed two peaks of 188 to 59 nanometers. Four days after packaging and pressurizing of the composition, foam was released from the aerosol can and light microscope observation revealed small population of ˜1 micron globules and substantial Brownian movement indicating that majority of oil droplets are of sub-micron or nano-scale.

6. Packaging and Pressurizing of the Nanoemulsion Composition

An emulsion (46 gram) was added into a 60 ml monoblock aluminum can. The can was closed with an aerosol valve and 4 gram of liquefied propellant (propane/butane mix) was added through the valve. The propellant can be any compressed and liquefied gas, currently used as aerosol propellant. The final concentration of propellant can vary from 3% to 25%.

### EXAMPLE 3

### Oil Bodies Based Foamable Compositions

| | NAT01 | NAT02 | NAT03 | NAT04 |
|---|---|---|---|---|
| | % w/w | % w/w | % w/w | % w/w |
| Natural Oleosomes (Natrulon | 30.00 | 30.00 | 30.00 | 30.00 |
| OSF)* | | | | |
| Hydroxypropylmethycellulose | 0.25 | 0.25 | — | — |
| (gelling agent) | | | | |
| Xanthan Gum (gelling agent) | 0.25 | 0.25 | — | — |
| Cocamide DEA (surfactant) | 1.00 | | 1.00 | |
| Polsorbate 20 (surfactant) | — | — | — | 1.00 |
| Water pure | 68.50 | 69.50 | 69.00 | 69.00 |
| | 100.00 | 100.00 | 100.00 | 100.00 |

Foam Properties

| | | | | |
|---|---|---|---|---|
| Foam Quality | E | E | E | G |
| Stability RT | Stable | Stable | Creaming | Creaming |
| | | | After 72 | After 72 |
| | | | Hr. | Hr. |

*Natrulon OSF is the trade name of Lonza Inc.

The production of the compositions NAT01 included the following steps:
1. Add the polymeric agents (Hydroxypropylmethycellulose and Xanthan Gum) to the Natrulon OSF at 50° C. and mix during 10 minutes while the preparation cools down to Room Temperature.
2. Add the Cocamide DEA with mixing.
3. Fill the composition aerosol canisters and add 8% of propellant.

The production of the compositions NAT02 included the following steps:
1. Add the polymeric agents (Hydroxypropylmethycellulose and Xanthan Gum) to the Natrulon OSF at 50° C. and mix during 10 minutes while the preparation cools down to Room Temperature.
2. Fill the composition aerosol canisters and add 8% of propellant.

The production of the compositions of NATO3 included the following steps:
1. Add the cool water to the Natrulon OSF and mix during 10 minutes.
2. Add the Cocamide DEA with mixing.
3. Fill the composition aerosol canisters and add 8% of propellant.

The production of the compositions of NAT04 included the following steps: 1. Add the cool water to the Natrulon OSF and mix during 10 minutes.
2. Add Polysorbate 20 with mixing.
3. Fill the composition aerosol canisters and add 8% of propellant.

### EXAMPLE 4

### Further Foamable Compositions Containing Oil Bodies

| | % w/w | % w/w |
|---|---|---|
| Caprylic/capric triglyceride (MCT oil) | 5.00 | — |
| Stearyl alcohol | 0.90 | — |
| Natrulon OSF* | 10.00 | 10.00 |
| Methylcellulose | 0.25 | 0.25 |
| Xanthan gum | 0.25 | 0.25 |
| PEG-40 stearate | 2.50 | 2.50 |
| Polysorbate 80 | 0.90 | 0.90 |
| Preservative | 0.50 | 0.50 |
| Purified water | to 100% | to 100% |
| Propellant | 8.00 | |

Formation properties

| Emulsion visual test | Uniform | Uniform |
|---|---|---|
| Viscosity (Spindle SC4-31)(cP) | 1,428 | 868.5 |
| Centrifugation (prior to propellant addition) | Stable | Stable |
| (10 min/3,000 rpm) | | |
| PH (direct, prior to propellant addition) | 6.04 | 6.72 |
| Foam Quality | G | E |
| Density | 0.0337 | 0.0339 |

What is claimed is:

1. A foamable oil in water emulsion composition comprising:
(a) an oil globule system, selected from
i. oil bodies, and
ii. sub-micron oil globules;
(b) about 0.1% to about 5% by weight of at least one stabilizing agent, selected from the group consisting of
i. a non-ionic surface-active agent, having an HLB value between 9 and 16,
ii. an ionic surfactant, and
iii. a polymeric agent;
(c) water; and
(d) a liquefied or compressed gas propellant at a concentration of about 3% to about 25% by weight of the total composition.

2. The foamable composition of claim 1, wherein the oil globule system consists of oil bodies; and the stabilizing agent consists of a polymeric agent.

3. The foamable composition of claim 1, wherein the oil globule system consists of oil bodies; and the stabilizing agent consists of an ionic surfactant.

4. The foamable composition of claim 2, further comprising a non-ionic surfactant having an HLB value between 9 and 16; and/or an ionic surfactant.

5. The foamable composition of claim 3, wherein the ionic surfactant is a zwiterionic surfactant.

6. The foamable composition of claim 4, wherein the ionic surfactant is a zwiterionic surfactant.

7. The foamable composition of claim 1, wherein the oil bodies are discrete oleaginous particles having a size ranging from about 1 to about 3 μm in at least one dimension.

8. The foamable composition of claim 1, wherein the oil bodies consist of triacyglycerols, surrounded by phospholipids and oleosins.

9. The foamable composition of claim 8, wherein the phospholipids are selected from the group consisting of phosphatidylethanolamine, phosphatidylcholine, lecithin, phosphatidylserine, phosphatidylglycerol and phosphatidylinositol.

10. The foamable composition of claim 1, wherein the oil bodies are derived from plant seeds.

11. The foamable composition of claim 10, wherein the plant is selected from the group consisting of almond (Prunus dulcis); anise (Pimpinella anisum); avocado (Persea spp.); beach nut (Fagus sylvatica); borage (also known as evening primrose) (Boragio officinalis); Brazil nut (Bertholletia excelsa); candle nut (Aleuritis tiglium); carapa (Carapa guineensis); cashew nut (Ancardium occidentale); castor (Ricinus communis); coconut (Cocus nucifera); coriander (Coriandrum sativum); cottonseed (Gossypium spp.); crambe (Crambe abyssinica) ; Crepis alpina; croton (Croton tiglium); Cuphea spp.; dill (Anethum gravealis); Euphorbia lagascae; Dimorphoteca pluvialis; false flax (Camolina sativa); fennel (Foeniculum vulgaris); groundnut (Arachis hypogaea); hazelnut (coryllus avellana); hemp (Cannabis sativa); honesty plant (Lunnaria annua); jojoba (Simmiondsia chinensis); kapok fruit (Ceiba pentandra); kukui nut (Aleuritis moluccana); Lesquerella spp., linseed/flax (Linum usitatissimum); macademia nut (Macademia spp.); maize (Zea mays); meadow foam (Limnanthes alba); mustard (Brassica spp. and Sinapis alba); oil palm (Elaeis guineeis); oiticia (Licania rigida); paw paw (Assimina triloba); pecan (Juglandaceae spp.); perilla (Perilla frutescens); physic nut (Gairopha curcas); pilinut (Canariuim ovatum); pine nut (pine spp.); pistachio (Pistachia vera); pongam (Bongamin glabra); poppy seed (Papaver soniferum); rapeseed (Brassica spp.); safflower (Carthamus tinctorius); sesame seed (Sesamum indicum); soybean (Glycine max); squash (Cucurbita maxima); sal tree (Shorea rubusha); Stokes aster (Stokesia laevis); sunflower (Helianthus annuus); tukuma (Astocarya spp.); tung nut (Aleuritis cordata); and vernolnia (Verzonia galamensis).

12. The foamable composition of claim 1, wherein:
i. The oil globule system consists of sub-micron oil globules; and
ii. The stabilizing agent consists of a surface-active agent, having an HLB value between 9 and 16.

13. The foamable composition of claim 12, wherein the sub-micron oil globules contain at least one organic carrier selected from the group consisting of a hydrophobic organic carrier, a polar solvent, an emollient and mixtures thereof.

14. The foamable composition of claim 13, wherein said submicron oil globules are the group consisting of (i) about 2% to about 5%; (ii) about 5% to about 10%; (iii) about 10% to about 20%; and (iv) about 20% to about 50%.

15. The foamable composition of claim 1, wherein the sub-micron oil globules have a number-average size of less than 1,000 nm.

16. The foamable composition of claim 1, wherein the sub-micron have a number-average size range, selected from (i) 40 nm to 1,000 nm. (ii) 40 nm to 500 nm; (iii) 40 nm to 200 nm; and (iv) 40 nm to 100 nm.

17. The foamable composition of claim 12, wherein the sub-micron oil globules have a number-average size range selected from the group consisting of (i) less than 500 nm; (ii) less than 200 nm; and (iii) less than 100 nm.

18. The foamable composition of claim 12, wherein the sub-micron oil globules are produced by high sheer homogenization.

19. The foamable composition of claim 1, further comprising about 0.1% to about 5% by weight of a foam adjuvant selected from the group consisting of a fatty alcohol having 15 or more carbons in their carbon chain; a fatty acid having 16 or more carbons in their carbon chain; fatty alcohols derived from beeswax and including a mixture of alcohols, a majority of which has at least 20 carbon atoms in their carbon chain; a fatty alcohol having at least one double bond; a fatty acid having at least one double bond; a branched fatty alcohol; a branched fatty acid; and a fatty acid substituted with a hydroxyl group and mixtures thereof.

20. The foamable composition of claim 1, wherein said foamable composition is substantially alcohol-free.

21. The foamable composition of claim 1, wherein the oil globules are present in an amount selected from the group comprising of (i) about 0.05% and about 2%; (ii) about 2% and about 5%; (iii) about 5% and about 12%; and (iv) about 12% and about 24%.

22. The foamable composition of claim 1, further containing at least one therapeutic agent.

23. The foamable composition of claim 22, wherein the therapeutic agent is selected from the group consisting of an anti-infective, an antibiotic, an antibacterial agent, an antifungal agent, an antiviral agent, an antiparasitic agent, an steroidal antiinflammatory agent, an immunosuppressive agent, an immunomodulator, an immunoregulating agent, a hormonal agent, vitamin A, a vitamin A derivative, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a disinfectant, an anesthetic, an antiallergic agent, an alpha hydroxyl acid, lactic acid, glycolic acid, a beta-hydroxy acid, a protein, a peptide, a neuropeptide, a allergen, an immunogenic substance, a haptene, an oxidizing agent, an antioxidant, a dicarboxylic acid, azelaic acid, sebacic acid, adipic acid, fumaric acid, a retinoid, an antiproliferative agent, an anticancer agent, a photodynamic therapy agent, an anti-wrinkle agent, a radical scavenger, a metal oxide (e.g., titanium dioxide, zinc oxide, zirconium oxide, iron oxide), silicone oxide, an anti wrinkle agent, a skin whitening agent, a skin protective agent, a masking agent, an anti-wart agent, a refatting agent, a lubricating agent and mixtures thereof.

24. The foamable composition of claim 22, wherein the therapeutic agent is selected from the components of the oil bodies or sub-micron globules.

25. The foamable composition of claim 22, wherein the therapeutic agent is suitable to treat a disorder, selected from a dermatological disorder, a cosmetic disorder, a gynecological disorder, a disorder of a body cavity, wound and burn.

26. The foamable composition of claim 1, wherein the oil globules are oil bodies and wherein the surface-active agent is a phospholipid stabilizing agent.

27. The foamable composition of claim 1, wherein the stabilizing agent includes a mixture of at least one non-ionic surface-active agent and at least one ionic surfactant in a ratio in the range of about 100:1 to 6:1.

28. The foamable composition of claim 1, wherein the stabilizing agent comprises a combination of a non-ionic surface-active agent and an ionic surfactant, at a ratio of between 1:1 and 20:1.

29. The foamable composition of claim 1, wherein the stabilizing agent is a non-ionic surface-active agent.

30. The foamable composition of claim 29, wherein said emulsion is an oil in water emulsion and wherein the HLB of said non-ionic surface active agent is between about 2 and about 9.

31. The foamable composition of claim 1, wherein the stabilizing agent is a polymeric agent selected from the group consisting of a water-soluble cellulose ether and naturally-occurring polymeric material.

32. The foamable composition of claim 31, wherein said water-soluble cellulose ether is selected from the group consisting of methylcellulose, hydroxypropyl cellulose, hydroxypropyl methylcellulose (Methocel), hydroxyethyl cellulose, methylhydroxyethylcellulose, methylhydroxypropylcellulose, hydroxyethylcarboxymethylcellulose, carboxymethylcellulose, carboxymethylhydroxyethylcellulose, xanthan gum, guar gum, carrageenin gum, locust bean gum and tragacanth gum.

33. The foamable composition of claim 1, wherein, upon release from the aerosol can creates a stable foam which is thermally stable, yet breaks under sheer force.

34. The foamable composition of claim 33, wherein the density of the foam is selected from (1) less than 0.12 g/mL; (2) the range between 0.02 and 0.12; (3) the range between 0.04 and 0.10; (4) the range between 0.06 and 0.10.

35. A method of treating, alleviating or preventing a disorder of the skin, body cavity or mucosal surface, wherein said disorder involves insufficient hydration of skin or a mucosal surface as one of its etiological factors, comprising:
administering topically to a subject having said disorder, a foamed composition comprising:
(a) an oil globule system, selected from
i. oil bodies; and
ii. sub-micron oil globules
(b) about 0.1% to about 5% by weight of at least one stabilizing agent, selected from the group consisting of
i. a non-ionic surface-active agent, having an HLB value between 9 and 16,
ii. an ionic surfactant, and
iii. a polymeric agent;
(c) surface-active agent;
(d) water; and
(e) a liquefied or compressed gas propellant at a concentration of about 3% to about 25% by weight of the total composition.

36. The method of claim 25, wherein the oil globule system consists of oil bodies and the stabilizing agent consists of a polymeric agent.

37. The method of claim 35, wherein the oil globule system consists of oil bodies and the stabilizing agent consists of an ionic surfactant.

38. The method of claim 35, wherein the composition further comprises about 0.1% to about 5% by weight of a foam adjuvant is selected from the group consisting of a fatty alcohol having 15 or more carbons in their carbon chain; a fatty acid having 16 or more carbons in their carbon chain; fatty alcohols, derived from beeswax and including a mixture of alcohols, a majority of which has at least 20 carbon atoms in their carbon chain; a fatty alcohol having at least one double bond; a fatty acid having at least one double bond; a branched fatty alcohol; a branched fatty acid; a fatty acid substituted with a hydroxyl group and mixtures thereof.

39. The method of claim 35, wherein the composition further comprises an active agent effective to treat a disorder and wherein the disorder is selected from the group consisting of a vaginal disorder, a vulvar disorder, an anal disorder, a disorder of a body cavity, an ear disorder, a disorder of the nose, a disorder of the respiratory system, a bacterial infection, fungal infection, viral infection, dermatosis, dermatitis, parasitic infections, disorders of hair follicles and sebaceous glands, scaling papular diseases, benign tumors, malignant tumors, reactions to sunlight, bullous diseases, pigmentation disorders, disorders of cornification, pressure sores, disorders of sweating, inflammatory reactions, xerosis, ichthyosis, allergy, burn, wound, cut, chlamydia infection, gonorrhea infection, hepatitis B, herpes, HIV/AIDS, human papillomavirus (HPV), genital warts, bacterial vaginosis, candidiasis, chancroid, granuloma Inguinale, lymphogranloma venereum, mucopurulent cervicitis (MPC), molluscum contagiosum, nongonococcal urethritis (NGU), trichomoniasis, vulvar disorders, vulvodynia, vulvar pain, yeast infection, vulvar dystrophy, vulvar intraepithelial neoplasia (VIN), contact dermatitis, osteoarthritis, joint pain, hormonal disorder, pelvic inflammation, endometritis, salpingitis, oophoritis, genital cancer, cancer of the cervix, cancer of the vulva, cancer of the vagina, vaginal dryness, dyspareunia, anal and rectal disease, anal abscess/fistula, anal cancer, anal fissure, anal warts, Crohn's disease, hemorrhoids, anal itch, pruritus ani, fecal incontinence, constipation, polyps of the colon and rectum.

40. A method of promoting the penetration of an active agent into the surface layers of the skin and mucosal membranes, comprising: apply a foamable composition to the surface layers of a stem or mucosal membrane, the foamable composition comprising:
(a) an oil globule system, selected from
i. oil bodies, and
ii. sub-micron oil globules;
(b) about 0.1% to about 5% by weight of at least one stabilizing agent, selected from the group consisting of
i. a non-ionic surface-active agent, having an HLB value between 9 and 16,
ii. an ionic surfactant, and
iii. a polymeric agent;
(c) water;
(d) an active agent; and
(e) a liquefied or compressed gas propellant at a concentration of about 3% to about 25% by weight of the total composition.

41. The method of claim 40, wherein the active agent is selected from the group consisting of an anti-infective, an antibiotic, an antibacterial agent, an antifungal agent, an antiviral agent, an antiparasitic agent, an steroidal antiinflammatory agent, an immunosuppressive agent, an immunomodulator, an immunoregulating agent, a hormonal agent, vitamin A, a vitamin A derivative, vitamin B, a vitamin B derivative, vitamin C, a vitamin C derivative, vitamin D, a vitamin D derivative, vitamin E, a vitamin E derivative, vitamin F, a vitamin F derivative, vitamin K, a vitamin K derivative, a wound healing agent, a disinfectant, an anesthetic, an antiallergic agent, an alpha hydroxyl acid, lactic acid, glycolic acid, a beta-hydroxy acid, a protein, a peptide, a neuropeptide, a allergen, an immunogenic substance, a haptene, an oxidizing agent, an antioxidant, a dicarboxylic acid, azelaic acid, sebacic acid, adipic acid, fumaric acid, a retinoid, an antiproliferative agent, an anticancer agent, a photodynamic therapy agent, an anti-wrinkle agent, a radical scavenger, a metal oxide (e.g., titanium dioxide, zinc oxide, zirconium oxide, iron oxide), silicone oxide, an anti wrinkle agent, a skin whitening agent, a skin protective agent, a masking agent, an anti-wart agent and a refatting agent.
